(19) United States
(12) Patent Application Publication
(10) Pub. No.: US 2026/0145343 A1
(43) Pub. Date: May 28, 2026

(54) MULTI-JAW GRASPER AND RELATED SYSTEMS AND METHODS

(71) Applicant: ProMedica Health System, Inc., Toledo, OH (US)

(72) Inventors: Sameh Almadani, Toledo, OH (US); James Savage, Toledo, OH (US)

(21) Appl. No.: 19/401,576

(22) Filed: Nov. 26, 2025

Related U.S. Application Data
(63) Continuation-in-part of application No. PCT/US2025/045702, filed on Sep. 10, 2025.
(60) Provisional application No. 63/692,829, filed on Sep. 10, 2024.

(51) Int. Cl. B25J 15/02; A61B 1/00; B25J 15/00; B25J 15/10

(52) U.S. Cl. CPC B25J 15/022; A61B 1/00147; B25J 15/0033; B25J 15/10

(57) ABSTRACT

A multi-jaw grasper is disclosed, with related systems and methods. An elongate structure extends from a handle having a control element to a grasping portion including an attachment segments and jaws. Each attachment segment is connected to a proximal end portion of a respective set of two of the jaws. The jaws contact one another at, and extending longitudinally outward from, a common location between the attachment segments. A linkage extending within the elongate structure connects the control element to a proximal end portion of at least one of the jaws such that movement of the control element causes movement of the jaws between a closed position where jaws extend alongside one another and an opened position where a distal end of the jaws are splayed apart.

### CROSS-REFERENCE TO RELATED APPLICATIONS

This application is a bypass continuation in part of PCT/US2025/045702 filed internationally on Sep. 10, 2025, which claims the benefit of U.S. provisional patent application Ser. No. 63/692,829 filed Sep. 10, 2024, the disclosures of each of the foregoing being hereby incorporated by reference as if fully restated herein.

### TECHNICAL FIELD

Exemplary embodiments relate generally to a multi-jaw grasper and related systems and methods, such as for grasping objects, removing foreign bodies, and for debulking of various lesions.

### BACKGROUND AND SUMMARY OF THE INVENTION

For a variety of reasons, patients occasionally develop an esophageal obstruction, such as where a food bolus, foreign body, or other object, sometimes relatively soft in nature (e.g., food) but not always (e.g., coin, pen, other foreign object), becomes impacted and requires removal. Sometimes, such as in the case of relatively soft objects, a preferred approach is to debulk the object and push it into the stomach. In other cases, such as foreign object, particularly which are relatively hard, grasping and removal is sometime needed. However, existing devices do not always provide for efficient removal or other manipulation. Improved devices that provide for more efficient grasping, debulking, removal, or other manipulation are needed.

A multi-jaw grasper, such as for grasping objects, such as for food bolus debulking and/or removal, foreign object grasping and/or removal, lesion debulking, combinations thereof, and the like, among other applications, along with related systems and/or methods are disclosed. The device may include a distal end having three jaws, which are movable between a closed position where the jaws extend alongside one another and form an at least partially enclosed capsule, such as to grasp, debulk, and/or capture an object, such as a relatively soft object (e.g., food bolus) and/or relatively hard object (e.g., foreign body), and an opened position where the jaws are splayed apart. The jaws may include pointed ends and/or serrated edges for grasping, tearing, and/or debulking the object. The jaws may be domed shaped, such as with a concave interior surface, to create an internal cavity, such as to accommodate some or all of the object. The jaws may be part of a movable portion of a grasping end portion of the device. The grasping portion may also include an attachment portion, and the jaws may be attached to the attachment portion by way of ball and socket type joints. The attachment portion may be relatively fixed.

In exemplary embodiments, without limitation, the jaws include a notch in a distal end portion thereof, thereby forming protrusions on either side thereof. Preferably, the distal end of the jaws are curved such that, when in the closed position, the jaws form two relatively sharp, curved protrusions at the end of either jaw, such as for grasping or tearing. In other exemplary embodiments, without limitation, the jaws include essentially the reverse. Openings are provided on either side of a curved end protrusion, thereby forming a claw like structure. Preferably, the curve end protrusions of the jaws come together at the distal end of the jaws when the jaws are in a closed position. This facilitates tearing or grasping. Serrations may be provided along lengthwise outer edges of the jaws, the serrations optionally being cooperative.

In exemplary embodiments, without limitation, two of the balls are located at each of the jaws, and two of the sockets are located at each of attachment segments of the attachment portion. While the term ball is sometimes used herein, the balls may be protrusions that are not necessarily perfectly spherical or round. Likewise, the sockets need not necessarily be perfectly spherical or round. Each of the jaws may be connected to two of the attachment segments such that each of the attachment segments has sockets connected to the balls of two different jaws. The attachment portion may include slots between each of the attachment segments which provide sufficient flexibility in the attachment portion to secure the jaws thereto, such as by allowing the balls to snap fit into the sockets.

Reinforcement members (e.g., rods) may be extended between, and secured to (e.g., by welding), adjacent ones of the attachment portions to assist with holding such adjacent attachment portions together.

A sleeve may, alternatively or additionally, be provided which is fixed atop some or all of the attachment portion, such as a base portion thereof (which may be recessed to accommodate the sleeve), to secure the same.

In exemplary embodiments, the jaws are placed adjacent to one another at the attachment portion such that originate from a common location. At least a proximal end portion of the jaws, such as a portion of the jaws extending at or near the common origin, may contact one another when the jaws are moved between the opened and closed positions, which may assist with preventing the individual jaws from slipping out of their respective sockets. The proximal end portion of the jaws is preferably rounded and includes a ridge which interacts with the ridges of the other jaws to facilitate such movement.

In exemplary embodiments, without limitation, the proximal end portion of each of the jaws includes one or more recesses or a through hole which attach to a mechanical linkage (e.g., rod(s), cable(s), member(s)) which extends through an elongate portion of the device attached to the attachment portion and extending to a handle assembly, to a control element at the handle assembly so that a user may control opening and/or closing of the jaws. A friction mechanism or stop may be provided to temporarily secure the jaws in a specific position.

The mechanical linkages may include a ball or other enlarged object beyond the through hole to reduce or eliminate pull through. Alternatively, or additionally, the mechanical linkages may be looped back and secured to themselves or otherwise include a change of direction for additional engagement.

In other exemplary embodiments, the mechanical linkage engages a distal end portion of each of the jaws, such as by extending along the rounded surface and/or ridge thereof where the jaws interact. A groove may be provided in the rounded surface thereof for the linkage (e.g., wire). A through hole may be provided so that the linkage extends to and within an interior of the jaws and is secured at a more distal location of the jaw. An aperture may be provided at the distal location of the jaw for securing the linkage. The aperture may extend to an outer surface of the jaw, such as to provide a direction change of the linkage. This may allow for an increase closing and/or grip strength of the jaws. Optionally, a support surface for the linkage is provided at the interior of the jaw.

The device may include visual indicators (e.g., radiopaque markers) at the jaws. The device may include sensors at the jaws for measuring position and/or feedback (e.g., pressures) during use. The device may be in electronic communication with one or more remote electronic devices to display various status information. The device may include, or be connected to, one or more energy sources, such as for providing radio frequency energy by way of the mechanical linkages to the jaws, which may allow for heating the objects during interaction, which may assist with breakdown, tearing, cauterizing, combinations thereof, or the like.

The disclosed devices, systems, and/or methods improve efficiency of object removal, which can improve patient outcomes, such as but not limited to, by increasing success of removal and/or reducing sedation times, among other advantages. While object removal is sometimes shown and/or discussed, the disclosed devices, systems, and/or methods may be used in other applications, such as with other types and kinds of objects, endoscopic necrosectomy and debridement in pancreatic necrosis or walled-off pancreatic necrosis (WOPN), biopsies, polyplectomy, combinations thereof, or the like, such as in or by way of the esophagus, or other parts of the body, such as in a human or animal patient.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical, similar, or equivalent features, and wherein:

FIG. 1 is a perspective view of an exemplary multi-jaw grasper with a grasping end portion in an opened position;

FIG. 2 is a detailed perspective view taken at Detail A of FIG. 1, with the grasping end portion in a closed position;

FIG. 3 is a further detailed perspective view taken at Detail B of FIG. 2, with the grasping end portion in the closed position;

FIG. 4 is the detailed perspective view of FIG. 3 with the grasping end portion in the opened position;

FIG. 5 is the front perspective view of the grasping end portion of FIG. 4 in the opened position;

FIG. 6 is a perspective view of an attachment portion of the grasping end portion of the device of FIGS. 1-5, illustrated in isolation to illustrate additional features thereof;

FIG. 7 is a rear perspective view of a portion of the grasping end portion of the device of FIGS. 1-6 in the opened position, partially illustrated a wireframe view to illustrate additional features thereof;

FIG. 8 is a perspective view of one of the jaws of the grasping end portion of the device of FIGS. 1-7, illustrated in isolation to illustrate additional features thereof;

FIG. 9 is a top view of an exemplary linkage and ball for use with the device of FIGS. 1-8;

FIG. 10 is a detailed perspective view of another exemplary embodiment of the grasping portion of the device of FIG. 1-9, illustrated in the closed position;

FIG. 11 is a perspective view of the attachment portion of the embodiment of FIG. 10 illustrated in isolation;

FIG. 12 is a perspective, partially wireframe view of the embodiment of FIGS. 10-11 in the open position;

FIG. 13 is a perspective view of a jaw of the embodiment of FIGS. 10-12 illustrated in isolation;

FIG. 14 is a perspective view of the embodiment of FIGS. 10-13;

FIG. 15 is a perspective view of the embodiment of FIG. 14;

FIG. 16 is a perspective view of the device of FIGS. 1-8 deployed within an exemplary endoscope device;

FIG. 17 is a detailed perspective view of the device of FIGS. 10-15 deployed within an exemplary endoscope device;

FIG. 18 is a plan view of an exemplary system for use with the device of FIGS. 1-15;

FIG. 19 is a flow chart with an exemplary method of using the device(s) of FIG. 1-15 and/or system of FIG. 17-18;

FIG. 20 is a detailed perspective view of the embodiment of FIG. 12 with the jaws in the closed position and the attachment portion illustrated in partial transparency;

FIG. 21 is a detailed side section view of the embodiment of FIG. 20 with the jaws in the open position;

FIG. 22 is a detailed perspective sectional view of the embodiment of FIG. 21 with the jaws in the closed position;

FIG. 23 is a detailed top view of the grasping portion of the device(s) of FIGS. 1-22 with an exemplary mechanical linkage arrangement;

FIG. 24 is a perspective view of the FIG. 23 embodiment;

FIG. 25 is a detailed view of FIG. 24;

FIG. 26 is a detailed top view of another exemplary embodiment of the grasping end portion of FIG. 23 with another exemplary mechanical linkage arrangement;

FIG. 27 is a perspective view of the FIG. 26 embodiment;

FIG. 28 is a detailed view of FIG. 26;

FIG. 29 is a detailed top view of another exemplary embodiment of the grasping end portion of FIG. 23;

FIG. 30 is a perspective view of the FIG. 29 embodiment;

FIG. 31 is another perspective view of the FIG. 29 embodiment;

FIG. 32 is a detailed top view of another exemplary embodiment of the grasping end portion of FIG. 23 with another exemplary mechanical linkage arrangement;

FIG. 33 is a perspective view of the FIG. 32 embodiment;

FIG. 34 is another perspective view of the FIG. 32 embodiment;

FIG. 35 is an end view of an exemplary jaw of the grasping end portion of FIGS. 32-34 illustrated in isolation;

FIG. 36 is a perspective view of an interior of the jaw of FIG. 35;

FIG. 37 is a side view of the jaw of FIGS. 35-36; and

FIG. 38 is a detailed side, partially transparent view of the grasping end portion of FIG. 32-34.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

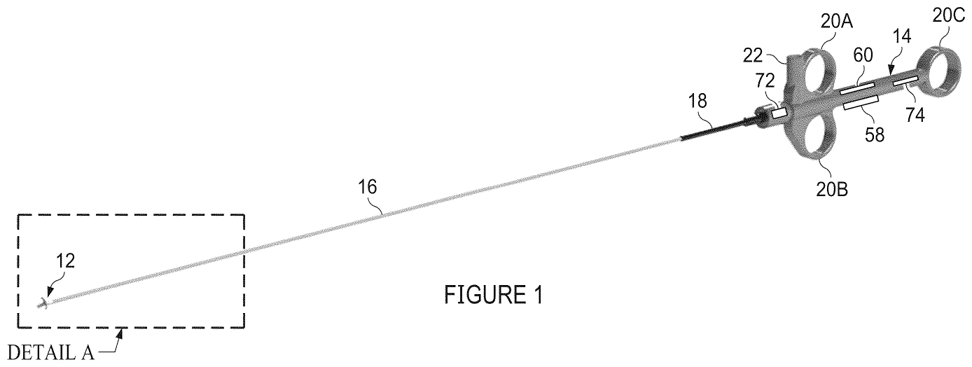

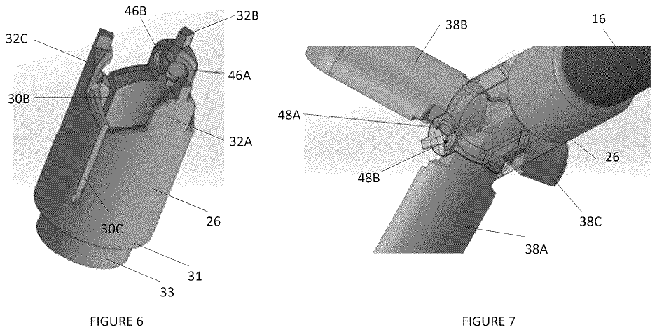

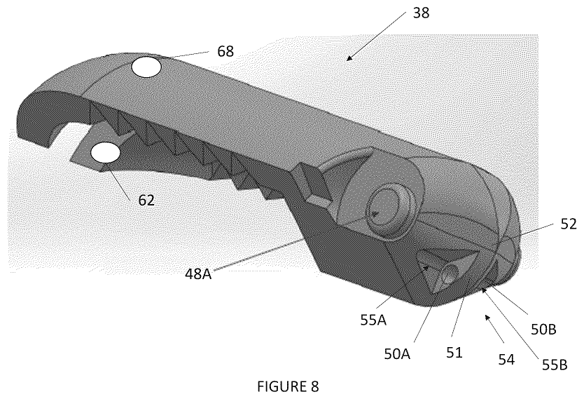

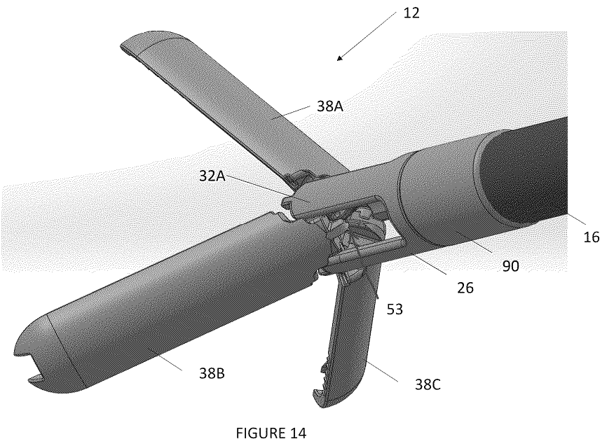

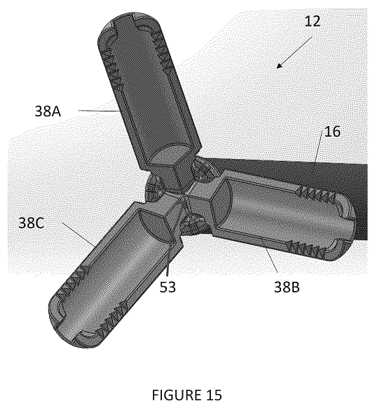

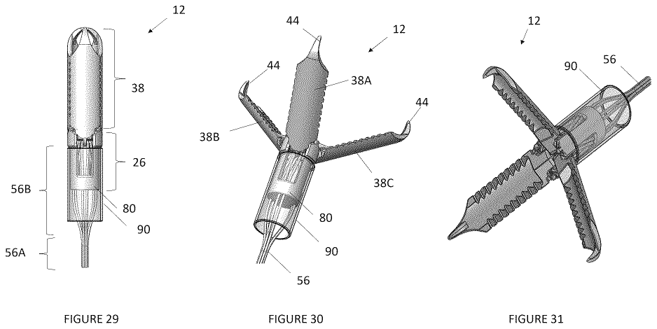

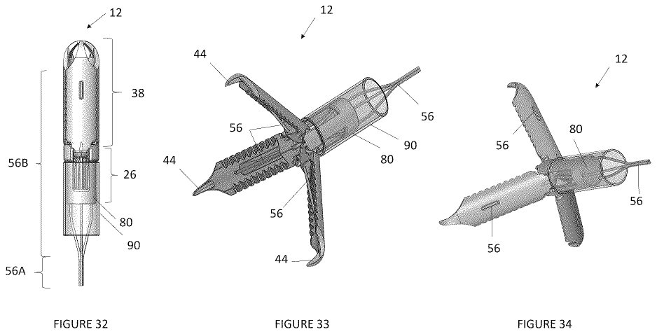

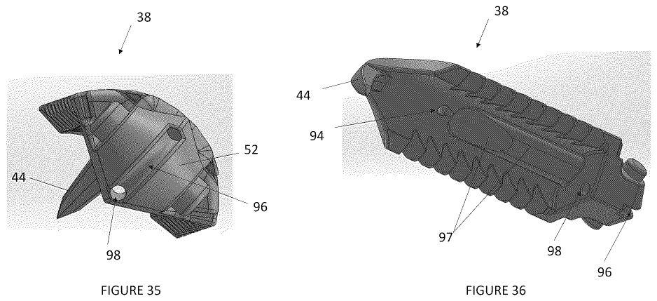

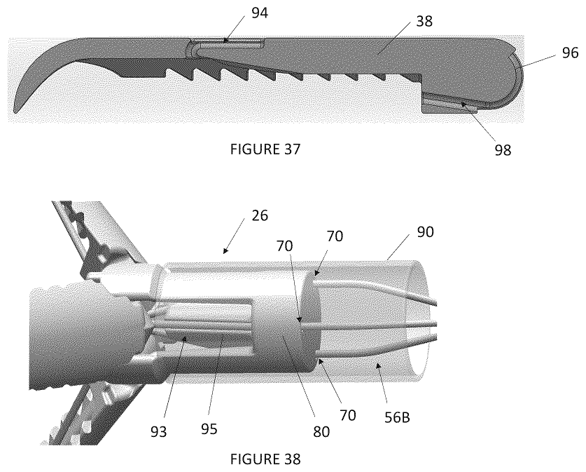

FIG. 1 through FIG. 15 and FIG. 20 through FIG. 38 illustrate a grasper 10 (may also be referred to herein as a grasper device, grasping device, device, grasper apparatus, grasping apparatus, and apparatus).

The grasper 10 may include a handle assembly 14. The handle assembly 14 may include a number of loops 20 for a user to place one or more fingers through. For example, without limitation, the handle assembly 14 may include a snare design with two adjacent loops 20A, 20B and a third loop 20C located at a proximal end of the handle assembly 14. While loops 20 may be shown and/or described, other features may alternatively or additionally be utilized to facilitate grip or manipulation of the device 10, such as but not limited to, hooks, surface texture, molded surfaces, protrusions, recesses, combinations thereof, or the like.

The handle assembly 14 may optionally include one or more connections 22, which may be in connection (e.g., fluid communication, connected passageway) with an interior of the handle assembly 14 and/or an elongated structure 16 extending therefrom. The connection(s) 22 may include luer connections, passageways for a guidewire, passageways for fluid introduction, combinations thereof, or the like.

Alternatively, or additionally, the connection 22 may provide electrical connection to the device 10. The connection 22 may comprise one or more electrically conductive materials. The connection 22 may comprise one or more insulating materials, such as about the one or more electrically conductive materials. The connection 22 may comprise one or more standardized plugs, wires, or other electrical connections. One or more power supplies may be connected to the connection 22 to provide energy to the device 10, such as further discuss herein. One or more wires may extend from the connection 22 through some or all of the device 10, such as to the grasping portion 12. Alternatively, or additionally, electrical connection may be made by way of the mechanical linkage(s) 56.

Various size, shape, and/or configuration handle assemblies 14 may be utilized.

The handle assembly 14 may include one or more control elements 58. In exemplary embodiments, without limitation, the control element 58 may include a slider which extends from a housing of the handle assembly 14 and is slidable, such as within a channel thereof, for manipulation by a user. Alternatively, or additionally, the control element 58 includes one or more of the loops 20, which may be movable. However, the control element 58 may include another manipulatable object, such as but not limited to, a trigger, knob, switch, combinations thereof, or the like.

A proximal end of the elongate structure 16 extends from, or through some or all of, the handle assembly 14. The elongate structure 16 may comprise a tube, which may be flexible and/or resiliently flexible, though such is not necessarily required. Other size and/or shape elongate structures 16 may be utilized, such as with varying levels of flexibility. Preferably, the elongate structure 16 is hollow, such as to accommodate one or more mechanical linkages 56, fluid from the connections(s) 22, combination thereof, or the like.

A proximal end portion of the elongate structure 16 may extend through and/or from a strain relief 18, which may extend from a distal end of, and or form part of, the handle assembly 14. The elongate structure 16 may be attached to the strain relief 18, or may extend therethrough. The strain relief 18 may provide enhanced rigidity to, limit flexibility of, and/or provide strain relief to, a portion of the elongate structure 16 passing therethrough.

The elongate structure may comprise one or more biocompatible polymers, metals, silicones, combinations thereof, or the like.

The device 10 includes a grasping portion 12. The grasping portion 12 is located at a distal end portion of the elongate structure 16, and may be attached thereto. The grasping portion 12 includes an attachment portion 26 and a movable portion 28. The attachment portion 26 may be entirely, or at least substantially (e.g., not including normal flexion or distortion during use), immobile relative to the moveable portion 28. The attachment portion 26 may be configured for attachment to the elongate structure 16. Preferably, a shape of the attachment portion 26 is complementary to a shape of the elongate structure 16. For example, without limitation, where the elongate structure 16 is tubular in shape, the attachment portion 26 may be shaped as a (preferably at least partially hollow) cylinder.

In exemplary embodiments, without limitation, some or all of an interior of the attachment portion 26 is hollow, such as to allow a distal end of the elongate structure 16 to extend, at least in part, therein. Alternatively, or additionally, a proximal end of the attachment portion 26 includes a mating portion 33, such as but not limited to a shelf, configured to receive a portion of the distal end of the elongate structure 16 thereover. For example, without limitation, the mating portion 33 may include a smaller diameter cylindrical shaped portion of the attachment portion 26 configured to snugly fit within the distal end portion of the elongate structure 16, which may also be cylindrical shaped, such as but not limited to in a friction fit, interference fit, compression fit, combinations thereof, or the like. Regardless, the elongate structure 16 may be secured to the grasping portion 12, such as by way of friction fit, adhesive, welding and/or swaging, fasteners (e.g., compression rings, clamps), combinations thereof, or the like. In other exemplary embodiments, without limitation, the elongate structure 16 and the grasping portion 12 are integrally formed.

The moveable portion 28 of the grasping portion 12 may comprise jaws 38. Preferably, the grasping portion 12 comprises three jaws 38A, 38B, and 38C, though other numbers such as two, four, or more jaws 38 may be utilized in other embodiments. In exemplary embodiments, without limitation, each of the jaws 38 comprises some or all of the same features (even if not specifically called out in the drawings) and/or is a same, or substantially the same (e.g., within 10%) size, shape, and/or other configuration. Some or all of the grasping portion 12, such as but not necessarily limited to the jaws 38, may comprise one or more metals, such as stainless steel.

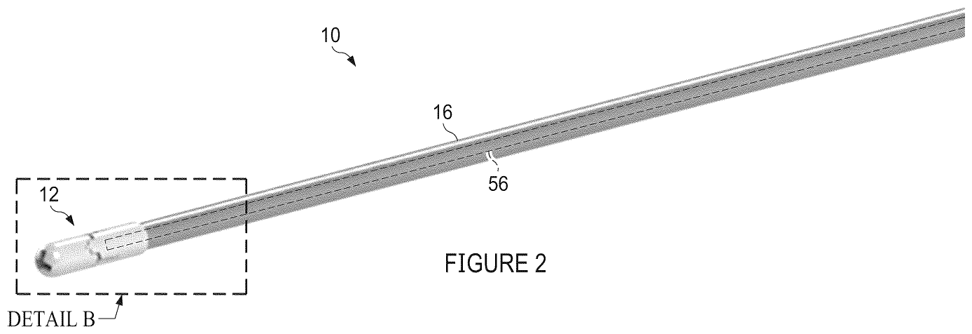

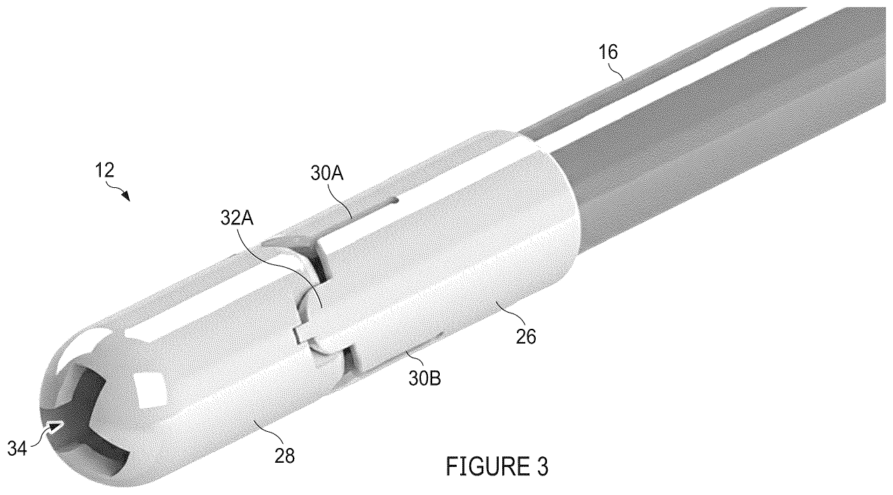

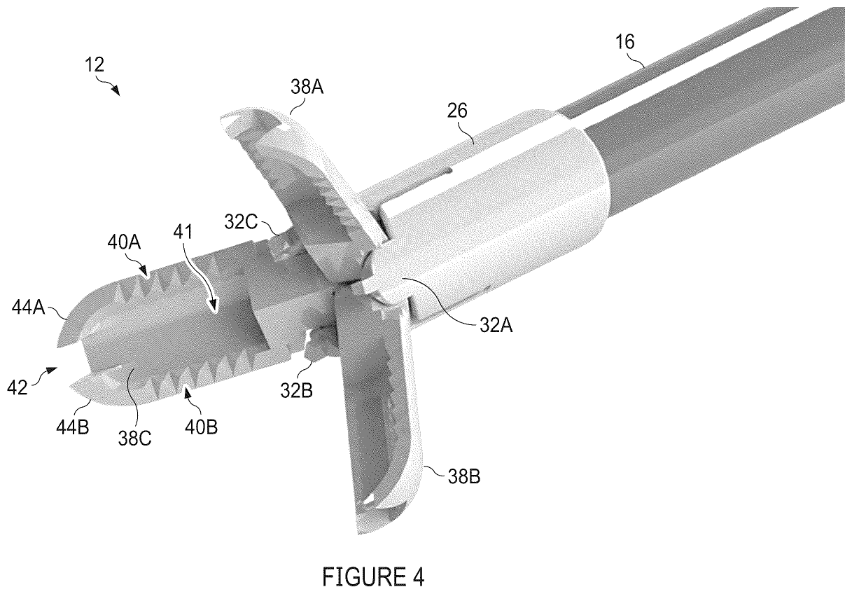

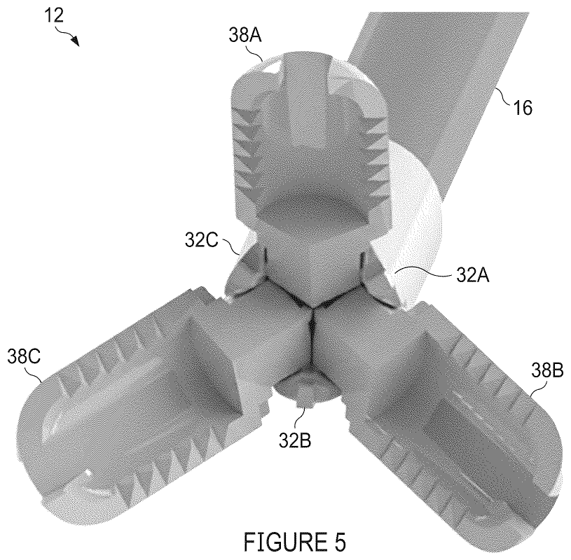

The jaws 38 of the grasping portion 12 may be configured for movement between a closed position (e.g., FIGS. 2-3) and an opened position (e.g., FIGS. 4-5). In the closed position, side edges of the jaws 38 may contact one another and/or extend alongside one another. In the closed position, the moveable portion 28 and the attachment portion 26 of the grasping portion 12 may be aligned such that they essentially form a unitary shape. In exemplary embodiments, without limitation, the grasping portion 12, when the jaws 38 are in the closed position, forms a generally cylindrical shape, such as with a rounded, distal end. In the opened position, the jaws 38 may be splayed apart such that a distal end of the jaws 38 are spaced apart from one another, and a proximal end portion 54 of the jaws 38 meet at the attachment portion 26. The proximal end portion 54 of the jaws 38 are, in exemplary embodiments, positioned to remain in contact at the attachment portion 26 when the jaws 38 are in both the opened and closed positions. The proximal end portion 54 of the jaws 38 may be located at least partially within the attachment portion 26.

Each of the jaws 38 may comprise an elongate domed shape, such as with a concave interior surface, where a peak of the dome extends along a longitudinal axis of the jaw 38. Each of the jaws 38 may include a rounded, distal end, preferably forming tips 44. Preferably, two tips 44A, 44B are formed in each jaw 38, and a channel 42 extends therebetween and into a space 41 formed by the domed shape of the jaw 38. In this way, when in the closed position, the distal end of the grasping portion 12 may define an opening 34, which may form a “Y” shape. In other exemplary embodiments, each of the jaws 38 may include a single tip 44 and/or rounded end, such as with or without any channel 42 and/or opening 34.

In other exemplary embodiments, without limitation, each of the jaws 38A, 38B, 38C comprise an elongate, curved distal end, preferably forming a tip 44A, 44B, 44C. Openings may be provided on either side of the tips 44 of the jaw 38. In this way, the tips 44 may each form a claw like shape, such as for tearing or grasping. The tips 44 of the jaws 38 may come together when the grasping end portion 12 is placed in the closed position, though physical contact between the tips 44 is not necessarily required.

Each of two longitudinally extending, interior facing edges 34 of the jaws 38 may include serrations 40. The serrations 40A, 40B may each extend along a respective side of the jaw 38. In exemplary embodiments, without limitation, the serrations 40 each extend along some or all of the length of the longitudinally extending edges 34 of the jaws 38. The serrations 40 are optionally cooperative.

The grasping portion 12 may be well suited for grasping, manipulating, debulking, capturing, combinations thereof, or the like of foodstuffs or other relatively soft objects or other relatively hard objects (e.g., foreign bodies), such as those impacted in a patient's esophageal area (e.g., food bolus, foreign body). For example, without limitation, some or all of the tips 44 and/or serrations 40 may allow grasping and/or manipulation. As another example, without limitation, the domed shape of the jaws 38 may allow grasped objects to be captured therein, such as for moving into a patient's stomach or removal from the body. While food boluses are sometimes discussed herein, other types and kinds of objects may be so grasped, manipulated, debulked, captured, combinations thereof, or the like by the device 10, such as in the esophageal area or elsewhere of a human or other type of animal. These may include foreign bodies (relatively soft or hard, e.g., coins, pens, paperclips, etc.), lesions, tissue, biological matter, non-biological matter, combinations thereof, or the like.

The jaws 38 may be movably connected to the attachment portion 26. In exemplary embodiments, without limitation, each of the jaws 38 comprise a first and second protrusion 48A, 48B, each of which mate with one of a first and second recess 46A, 46B located at the attachment portion 26. Thus, where three jaws 38 are utilized, six total protrusions 48 and recesses 46 may be provided, by way of non-limiting example. The protrusions 48 and the recesses 46 may provide ball and socket type jointing, with the protrusions 48 acting as the balls and the recesses 46 acting as the sockets, sometimes being referred to as such herein. The protrusions 48 may be part of the proximal end portions 54 of the jaws 38. Recessed areas may be provided about the protrusions 48, such as to facilitate movement of the jaws 38.

The arrangement of the protrusions 48 and recessed 46 may be opposite in other embodiments, such as with the protrusions 48 on the attachment portion 26 and the recesses on the jaws 38.

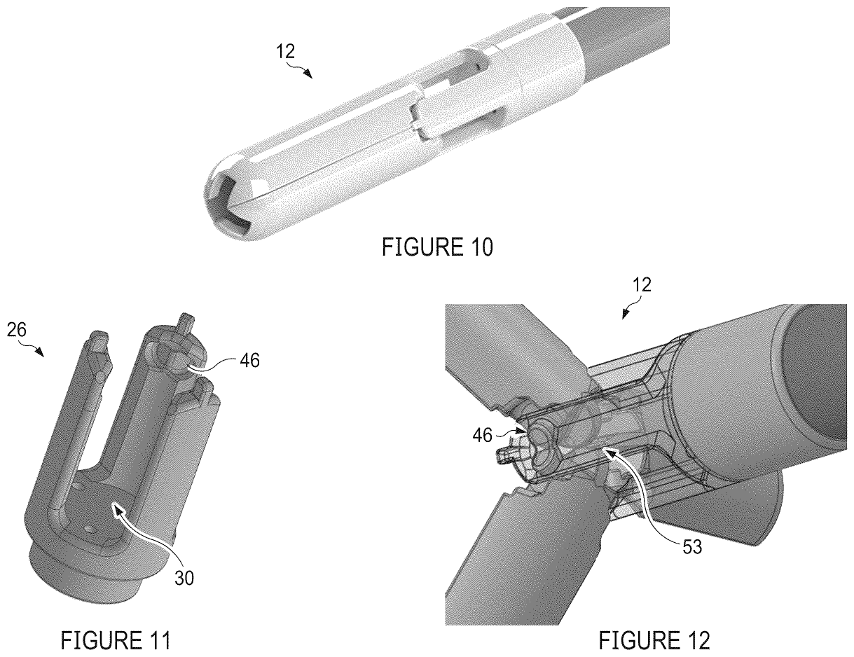

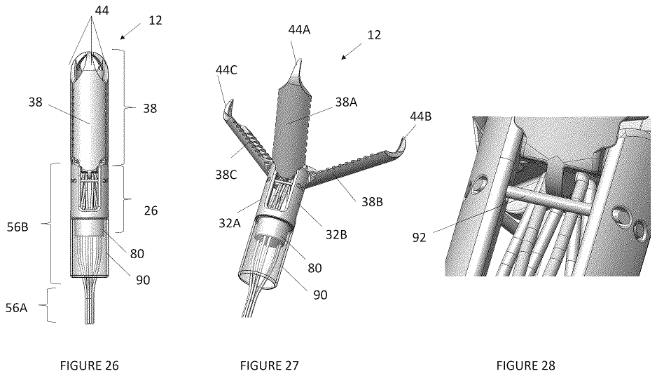

The attachment portion 26 may comprise a number of attachment segments 32. The attachment segments 32 may be interconnected by way of a common portion 31 of the attachment portion 26. The common portion 31 may comprise, by way of non-limiting example, a cylindrical shape. The recesses 46 may be located, preferably in pairs, at each of the attachment segments 32. Each of the attachment segments 32 are connected to two adjacent ones of the jaws 38 in exemplary embodiments. The number of attachment segments 32 may be equal to the number of jaws 38. A slot 30 may extend in the attachment portion 26 between each of the attachment segments 32, such as to define, at least in part, the attachment segments 32 and allow some flex during installation and/or use. The common portion 31 may be a portion of the attachment portion 26 without the slots 30, in exemplary embodiments. Thus, where three jaws 38 are utilized, three attachment segments 32A, 32B, 32C may be provided, with a slot 30A, 30B, 30C between each. This may provide flexibility at the attachment segments, and thereby may allow a snap or friction fit between the jaws 38 and the attachment portion 26, preferably by pushing each of the jaws 38 (together or separately) into the attachment portion 26, or vice-versa, until the protrusions 48 are received into (e.g., by friction and/or snap fit) the recesses 46. Optionally, such as illustrated with particular regard to FIGS. 26-28, a reinforcement member 92 is provided which connects adjacent attachment segments 32A, 32B, 32C. The reinforcement member 92 may comprise a rod which is welded or otherwise attached to the adjacent attachment segments 32A, 32B, 32C. Such as shown with particular regard to at least FIGS. 10-12 and 14, the slots 30 may be of various size, such as to accommodate various size jaws 38 and/or allow flexibility and movement of the jaws 38. For example, without limitation, such slots 30 may be large enough to form a window or opening, and may be referred to as such at times herein. This may allow ease of access for manufacture and servicing, such as but not limited to, attachment, replacement, and/or repair of the linkage(s) 56.

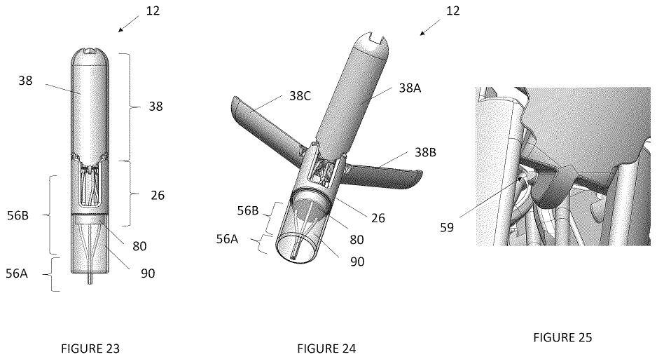

A sleeve 90, such as illustrated with particular regard to FIGS. 23-24, may be positioned over at least a portion of the attachment portion 26, though such is not required. In exemplary embodiments, the sleeve 90 extends over at least a portion of a length of the slots 30 after the jaws 38. The sleeve 90 may be positioned at the attachment portion 26 after the jaws 38 are secured at the attachment portion 26 to further secure the jaws 38 at the attachment portion 26 by preventing or inhibiting flexing of the attachment segments 32. The sleeve 90, where utilized, may be glued, welded, bonded, or the like to secure it in place.

The sleeve 90, where utilized, may secure the attachment portion 26 to the elongated structure 16. A base portion 80 of the attachment portion 26 may be recessed relative to the attachment segments 32 in exemplary embodiments to receive the sleeve 90.

In other exemplary embodiments, without limitation, the protrusions 48 are provided in the form of pins, and the recesses 46 are provided in the form of corresponding apertures, such that the protrusions 48 and the recesses 46 form pin type joints. Regardless, the protrusions 48 and recess 46 facilitate movement of the jaws 38 between the opened and closed positions.

A reflex angle may be formed between inward facing surfaces of the recesses 46 of each of the attachment segments 32 to accommodate each of the jaws 38. Where a different number of jaws 38 are utilized, the size and shape of the jaws 38, and thus the angle between the inward facing surfaces of the recesses 46, may be adjusted.

Each pair of recesses 46 or a given attachment segment 32 may be at least partially co-extensive in scope, such that they form a singular, connected space with two distinct portions, by way of non-limiting example. In other exemplary embodiments, the recesses 32 of a given attachment segment 32 may be spaced apart from one another.

A proximal end portion 54 of the jaws 38 may include a rounded surface 52 located at a proximal end of the respective jaw 38. The rounded surface 52 may be dome shaped at some or all of an upper half thereof, and may comprise a ridge 51 extending therefrom, such as at some or all of a lower half thereof. In this regard, the ridges 51 of the jaws 38 contact one other, such as along upper surfaces thereof, to secure and allow movement of the jaws 38. This is particularly well illustrated in at least FIG. 12, by way of non-limiting example. The area of interaction is generally indicated at item 53, which is also a common location where the jaws 38 meet and extend from. The proximal end portion 54 of each of the jaws 38 may include recessed areas 55A, 55B, such as in the lower half of the dome shape, which may define, at least in part, the ridge 51. Each of the recessed areas 55 may comprise and/or define, at least in part, a surface in which further recesses 50A, 50B are located. Alternatively, a single, through hole 50 may be utilized which extends through the ridge 51. The recess(es)/through hole(s) 50 may be part of the proximal end portion 54 of the jaws 38.

The recess(es)/through hole(s) 50 may be connected to the mechanical linkage(s) 56. The mechanical linkage(s) 56 may comprise a member or members, a rod or rods, wire or wires (e.g., braided), and/or other mechanical linkages, which preferably extend from, and are connected to, the recess(es)/through hole(s) 50, and through some or all of the elongate structure 16, which may be at least partially hollow to accommodate the same.

In exemplary embodiments, the mechanical linkage 56 includes a first portion 56A having multiple elements (e.g., wires; sometimes each individual element being referred to herein as a linkage) provided together and/or a unitary linkage (e.g., wire) which extends along a length of the device 10, such as from the handle assembly 14 to the grasping portion 12, such as to a position adjacent to the jaws 38. The linkage 56 may thereafter divide and/or the various individual linkages (e.g., wires) thereof may separate, such as to attach to each of the jaws 38, respectively. This is sometimes referred to as the second portion 56B of the linkage and is illustrated with particular regard to at least FIGS. 23-25. The first portion 56A may include bonded wires (e.g., by welding) adjacent wires (e.g., by ties, braiding, or the like), combinations thereof, or the like. Alternatively, the first portion 56A may comprise a unitary wire or other element. The second portion 56B may included separated, individual ones of the first portion 56A (e.g., separated ones of the bundled and/or braided wires) or individual linkages connected to a unitary first portion 56A, by way of non-limiting example. Notably, the first and second portions 56A, 56B may be integrally formed elements which are together in the first portion 56A and separated (at least for a length of the device) at the second portion 56B, or separate but connected elements.

As illustrated with particular regard to FIGS. 26-28, in other exemplary embodiments, the second portion 56B of the mechanical linkage 56 may include individual wires or other linkages which loop through the through holes 50 of the jaws 38 and connect back to the first portion 56A of the mechanical linkage 56, such as by bonding, attachment, combinations thereof, or the like. In this way, the individual portions of the linkage 56 may be looped through the holes 50 and secured to themselves, for example.

Pushing and/or pulling the mechanical linkage 56 may be configured to apply forces to the lower half of the proximal end portion 54 of the jaw 38 where the recess(es)/through hole(s) 50 are located, thereby causing movement of the jaw or jaws 38 between the opened and/or closed position, such as by frictional co-engagement with one another at the rounded surface 52 and/or by the rounded surface 52 along another surface.

A separate mechanical linkage 56 may be utilized for, and be connected to, each of the jaws 38. In other exemplary embodiments, a single mechanical linkage 56 is used for, and is connected to, all of the jaws 38. However, in yet other exemplary embodiments, a single mechanical linkage 56 is connected to a single one of the jaws 38, but is utilized to move all of the jaws 38, such as by frictional engagement with one another. In yet other exemplary embodiments, a single mechanical linkage 56 may have multiple ends, each of which attach to a given one of the jaws 38. This may be accomplished in the form of interconnected members, a braided wire with an end strand or strands being connected to a respective jaw 38, combinations thereof, or the like. However, a braided wire may be utilized even where the linkage 56 is connected to only a single one of the jaws 38.

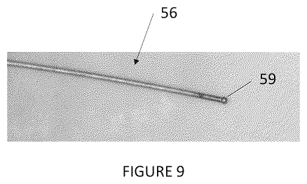

In exemplary embodiments, without limitation, the mechanical linkage(s) 56 is/are secured at the recess(es)/through hole(s) 50 by swaging the linkage(s) 56 to the jaw(s) 38 thereat. Alternatively, or additionally, a ball or other shaped tip 59 is formed at a distal end of the linkage(s) 56 that cannot be pulled through the through hole after passing the linkage 56 though the through hole. An exemplary linkage 56 and tip 59 is provided in FIG. 9, by way of non-limiting example. Alternatively, or additionally, the linkage(s) 56 may change direction after passing through the recess(es)/through hole(s) 50 to assist with preventing pull-through.

As illustrated with particular regard to FIGS. 26-28, in other exemplary embodiments, the second portion 56B of the mechanical linkage 56 may include individual wires or other linkages which loop through the through holes 50 of the jaws 38 and connect back to the first portion 56A of the mechanical linkage 56 (e.g., to themselves or to other individual elements of the linkage 56).

In exemplary embodiments, without limitation, the tip 59 is formed by heating a tip of the linkage 56, an approximately 0.006 inch diameter wire in this exemplary embodiment, such as using a laser, to create an approximately 0.009 inch diameter ball-shaped tip 59. The ball-shaped tip 59 may be formed by laser welding, mechanically swaging, combinations thereof, or the like. The size and shape of the tip 59 is exemplary, though preferred. Even where the term “ball” is used, the tip 59 need not be perfectly spherical. The nature of the linkage 56 as a wire is also exemplary, though preferred. A common wire with a single tip 59 may be threaded through the through holes of each of the jaws 38, a single wire with a single tip 59 may be threaded through the through hole of a single one of the jaws 38, or by separate wires, or wire ends from a common wire, and tips 59 may be utilized for each of the jaws 38, by way of example. The foregoing, including but not limited to the dimensions thereof, being exemplary and not intended to be limiting. Other dimensions and/or techniques, including but not necessarily limited to dimensions within (+/−) 200% of any specific stated dimension may be utilized.

The control element 58 may be connected, such as via the mechanical linkage 56 and/or other intermediary mechanical components to the grasping portion 12, and specifically jaws 38 thereof. The intermediary mechanical components may include, for example without limitation, pulleys, levers, gears, threaded rods, combinations thereof, or the like. These intermediary mechanical components may allow movement of the control element 58 to result in movement of the jaws 38. The intermediary mechanical components may optionally allow for amplification or de-amplification of movement of the control element 58 at the jaws 38. For example, without limitation, this may allow relatively large movement at the control element 58 to provide relatively fine movement at the jaws 38, or vice-versa.

Optionally, the control element 58 may include a friction mechanism or stop 72 which prevents or inhibits movement of the control element 58 when engaged, or such a friction mechanism or stop 72 may be provided at the handle 14. This may allow jaws 38 to be temporarily secured in various positions. Such friction mechanisms or stops 72 may include straps, knobs, levers, screws, springs, clamps, protrusions, recesses, notches, combinations thereof, or the like. The friction mechanisms or stops 72 may be configured to, when activated, frictionally engage the control element 58 and/or mechanical linkage 56. By way of non-limiting example, the friction mechanisms or stops 72 may include a knob connected to a screw, which when turned, places compressive forces on the linkage 56. As another example, without limitation, the friction mechanisms or stops 72 may include a series of notches for the control element 58 to be secured within, such as along the channel. As yet another example, without limitation, the friction mechanisms or stops 72 may include a spring or clamp for placing compressive forces on the mechanical linkage 56. This may allow the jaws 38 to be temporarily secured in various positions, such as the closed position, or partially closed position, with an object therein or grasped thereby, which may aid with facilitating tearing and removal. Optionally, a spring or other mechanism may be utilized within the handle 14, such as in connection with the mechanical linkage 56 and/or loops 20 to limit an amount of force that can be applied to the mechanical linkage 56 and therefore the jaws 38. For example, without limitation, one or more springs or other mechanical components may be interposed between one or more of the loops 20 and the mechanical linkage 56 such that movement of the loops 20 causes corresponding movement of the spring, thereby linking the ultimate force transmitted to the mechanical linkage 56, and therefore the jaws 38.

As illustrated with particular regard to FIGS. 7, 12, 14, and 20 in exemplary embodiments, without limitation, the protrusions 48 of the jaws 38 are each placed adjacent to one another, such as within a respective socket 46 at the attachment portion 26. The sockets 46 may be positioned adjacent to one another, and optionally connected, such that the jaws 38 originate from a common location as generally indicated at item 53 of FIGS. 9, 12, 14-15, and 20. At least a portion of the jaws 38, such as some or all of the proximal end portions 54 thereof, may contact one another, which may assist with preventing the individual jaws 38 from slipping out of their respective sockets 46.

In exemplary embodiments, the sockets 46 are positioned to cause the protrusions 48 of adjacent jaws 38 to contact one another when installed. In this way, the sockets 46 may ride along one another as the jaws 38 are moved. This contact between the sockets 46 may further constrain and guide movement and/or position of the jaws 38. This arrangement and contact is particularly well illustrated with protrusions 48A of jaw 38A and protrusion 48B of jaw 38B of FIGS. 7, 12, and 20.

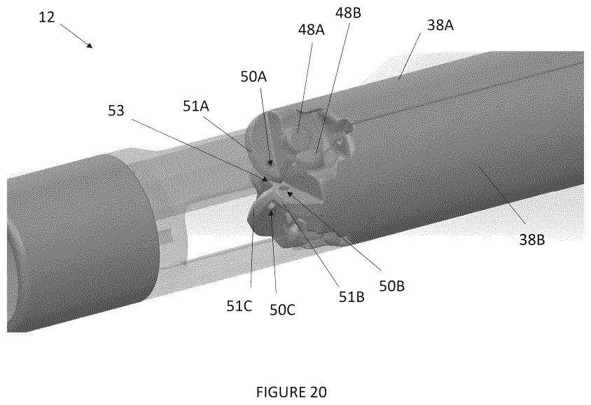

As particularly visible in FIGS. 12 and 20, the ridges 51 of the jaws 38 may contact one another to facilitate movement of the jaws 38 as generally indicated at item 53 of FIGS. 12 and 20, by way of example. The ridges 51 may form part of the rounded surface 52 and/or match the same by curvature and smooth transition. As particularly well illustrated in FIGS. 7 and 20, by way of example, where three jaws 38A, 38B, 38C are utilized, each of the ridges 51A, 51B, 51C may interact along a length thereof at the common location 53. The ridges 51 may move along one another as the jaws 38 are moved between the open and closed position. In this way, the ridges 51 may constrain and guide movement and/or position of the jaws 38.

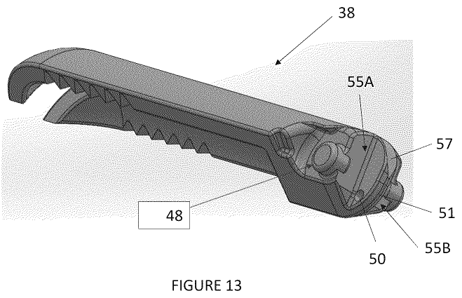

As illustrated with particular regard to at least FIG. 13, the recessed areas 55A, 55B may be provided at all, or substantially all (e.g., at least 80%) of the proximal end portion 54 of some or all of the jaws 38, such that the ridge 51 may extend over some or all the entire proximal end portion 54 of the respective jaw 38 and a separate dome shaped rounded surface portion is not required.

A smoothed surface 57 may be provided over part of the proximal end portion 54, though such is not required. The smoothed surface 57 may not be in contact with other jaws 38 in exemplary embodiments, and thereby may not form part of the ridge 51 or at least part of the ridge 51 which contacts the other jaws 38.

Each of the jaws 38 may be connected to a single mechanical linkage 56, such as to facilitate common movement. Alternatively, each of the jaws 38 may be connected to a respective mechanical linkage 56 to facilitate individual control. In other exemplary embodiments, without limitation, the jaws 38 may be collectively controlled by a single mechanical linkage 56 connected to a single one of the jaws 38. In such embodiment, by way of non-limiting example, only one of the jaws 38 may include the recess(es)/though hole(s) 50. The remaining jaws 38 may include the rounded surface 52 and/or ridges 51. However, all jaws 38 may be manufactured identically for simplicity, even if not separately controlled. Frictional engagement between the jaws 38, such as at the rounded surface 52 and/or ridges 51, may cause movement of the other jaws 38 upon movement of the one of the jaws 38 to which the mechanical linkage 56 is connected.

As illustrated with particular regard to FIG. 20, each of the jaws 38A, 38B, 38C may include a corresponding through hole 50A, 50B, and 50C below the ridge 51A, 51B, 51C thereof, at the proximal end portion 54 thereof.

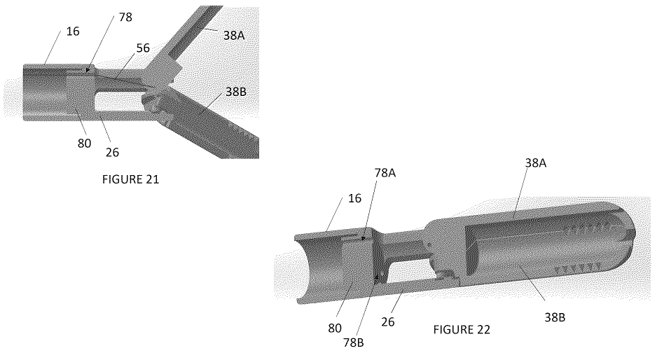

As illustrated with particular regard to FIGS. 21-22, one or more channels 78 may be provided in a base portion 80 of the attachment portion 26. In exemplary embodiments, one of the channels 78 is provided for each of the linkages 56, or at least the second portions 56B thereof. In this way, where the linkage 56 comprises, for example, multiple wires (e.g., one for each of the jaws 38), a respective one of the wires may be passed through each of the channels 78 to provide and maintain separation and/or mechanical leverage. The base portion 80 may be sized to fit within and/or provide a cap extending overtop the distal end of the elongate structure 16. In this way, the base portion 80 may block the distal end of the elongate structure 16, which may be hollow. The base portion 80 may be secured to the elongate structure by adhesive, welding, bonding, mechanical connection (e.g., interference fit, friction fit), combinations thereof, or the like.

The channel(s) 78 may be through holes in the base portion 80 for accommodating the mechanical linkage 56. Where a single mechanical linkage 56 is utilized, a single channel 78 may be provided. Where multiple mechanical linkages 56, or multiple ends thereof (e.g., of a braided wire), are utilized, multiple channels 78A, 78B, 78C (78C not illustrated) may be utilized. Preferably, the channel(s) 78 are located adjacent an outer edge of the base portion 80 (e.g., the outer circumferential surface thereof), thereby offsetting the channel(s) 78 from the common location 53 and/or holes 50 where the linkage(s) 56 attach to the jaws 38. This may increase the moment arm experienced at the jaws 38, allowing easier movement of the jaws 38. Preferably, a channel 78 is provided for each of the jaws 38, and particularly each of the jaws 38 having one of the holes 50 for connection to the linkage(s) 56. The portion of the mechanical linkage 58 extending through the through hole 50 of the jaw 38 may include the ball shaped tip 59 for preventing retraction of the linkage 58 back through the hole 50 and/or causing engagement with the jaw 38, in exemplary embodiments, without limitation.

In the embodiments of, for example, FIGS. 26-28 where the second portion 56B of the mechanical linkage 56 loops back, separate channels 78 may optionally be employed for each direction of travel.

In exemplary embodiments, such as illustrated with particular regard to FIGS. 32-38, the linkage 56, and particularly the second portion 56B thereof, may extend further along the jaws 38, such as to a distal half and/or end portion thereof, whereby it may be secured. This may increase mechanical leverage and corresponding grip force, such as at the tips 44.

In such embodiments, by way of example and without limitation, the linkages 56 (e.g., wires) need not necessarily pass through the through holes 50 of the jaws 38, the through holes 50 thereby being optional, but instead may extend along the curved proximal end surfaces 52 of the jaws 38. In such embodiments, the linkages 56 may extend along the ridges 51 thereof. In other such exemplary embodiments, the curved surfaces 52 may include a groove 96 for accommodating the linkage 54. The groove 96 may be provided at an exterior surface of the rounded proximal end portion of the jaws 38.

An aperture 98 may be provided at or near an end of the groove 96. The aperture 98 preferably passes through the jaw 38 and thereby allows the linkage 56 to extend to an interior facing side of the jaw 38. The use of the aperture 98 may assist with preventing the linkage 56 from bowing outward when advanced distally to open the jaws 38. Optionally, the linkage 56 extends to, and is anchored at, a recess 94 at a distal end portion of the jaw 38. The recess 94 preferably extends through the jaw 38 to an outward facing surface thereof, thereby allowing the linkage 54 to reverse direction before being secured. This may assist with jaw 38 operations.

A support surface 97 may optionally be provided within the jaw 38 to support the linkage 54. The support surface 97 may protrude from an interior facing surface of the jaw 38 and may optionally comprise one or more ramped surfaces.

In such embodiments, each linkage 56 preferably extends from one of the channels 78 opposing (e.g., most distal to) the respective jaw 38 so that each linkage 56 wraps around the curved proximal end surfaces of the jaws 38 (e.g., via ridges 51 and/or grooves 96) thereof so as to act as a pulley and increase leverage.

In such embodiments, by way of example and without limitation, the attachment portion 26 includes a guide support 95 on an interior thereof. The guide support 95 may be a generally cylindrical or frustoconical shaped protrusion from the base 80 which supports the linkages 56, especially but not necessarily as they traverse from one of the channels 70 to an opposing one of the jaws 38. The size and/or shape of the guide support 95 is exemplary. Grooves 93 may be provided within the guide support 95 for each of the linkages 56. The guide support 95 may support and/or guide the end portions of the linkage 56B, such as to prevent rubbing, to provide appropriate forces at the jaws 38 for movement, and/or to prevent the linkages 56 from bowing inward when advanced distally to open the jaws 38.

The jaws 38 may be identical (at least within manufacturing tolerances) in exemplary embodiments. This may allow the jaws 38 to be opened and closed in an at least substantially symmetrical manner. In other embodiments, without limitation, the jaws 38 may be different. For example, the location of the holes 50 and/or size and shape of the ridges 51 or proximal end portions 54 may vary to offset the jaws 38 so that they may be open and closed in an unsymmetrical manner.

Regardless, in exemplary embodiments, without limitation, the mechanical linkage(s) 56 may extend to the handle assembly 14, such as to the control element 58. While mechanical linkages are sometimes shown and/or described herein, those of skill in the art will appreciate that electronic, motorized control (e.g., by wired or wireless connection) may be utilized. Alternatively, or additionally, the control element 58 may optionally be located outside of the handle assembly 14.

The control element 58 and/or mechanical linkage 56 may be configured to provide push and pull control, such as to require manual actuation to move the jaws 38 between the opened and closed position. In other exemplary embodiments, without limitation, the jaws 38 may be biased in the opened and/or closed position, such as by way of one or more springs, such that manual actuation is required to move the jaws 38 to a respective one of the opened and closed positions.

Optionally, the handle assembly 14 may comprise one or more displays 60 (e.g., electronic display, gauge, indicators along the control element 58, combinations thereof, or the like) indicating the status of the device 10, such as the opened and/or closed position of the jaws 38, or degree/amount of opened of the jaws 38. For example, without limitation, as the control element 58 is actuated and/or the mechanical linkage 56 is moved, the displays 60 may be updated. In this way, the user may be made aware of the device's 10 status.

Alternatively, or additionally, the grasping portion 12 may comprise one or more sensors 62, such as but not limited to, temperature sensors, harness sensors, pressure sensors, force sensors, gyroscopes, imaging devices (e.g., camera), combinations thereof, or the like, data from which may be presented at the same or different displays 60. In this way, the user may be made aware of the device's 10 status or the status of the object therein, by way of non-limiting example.

In exemplary embodiments, without limitation, each of the jaws 38 may have a longitudinal length of between 5-20 mm in longitudinal length, preferably between 6 and 17 mm, and more preferably about 15 mm (+/−1 mm, e.g., as shown in FIGS. 10-15 and 17), though other sizes may be utilized. The jaws 38 may open such that a maximum distance between the distal ends of each of the jaws 38 is between 10 and 25 mm, preferably between 16 and 20 mm. These dimensions are exemplary and not intended to be limiting. Overall length of the device 10 may be between 50 and 200 cm in exemplary embodiments, more preferably about 100 mm (+/−10mm), by way of non-limiting example.

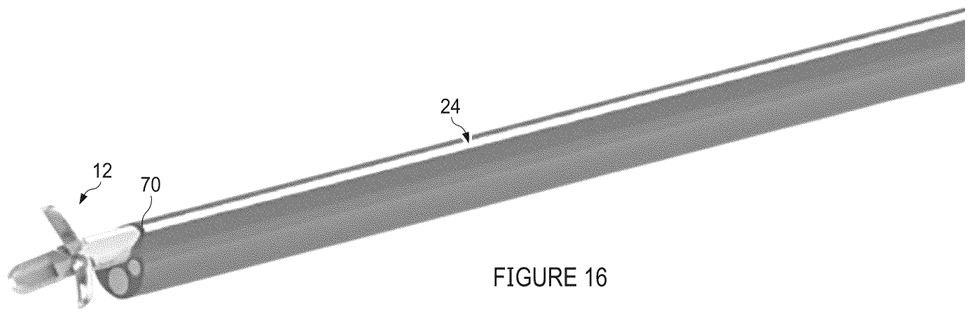

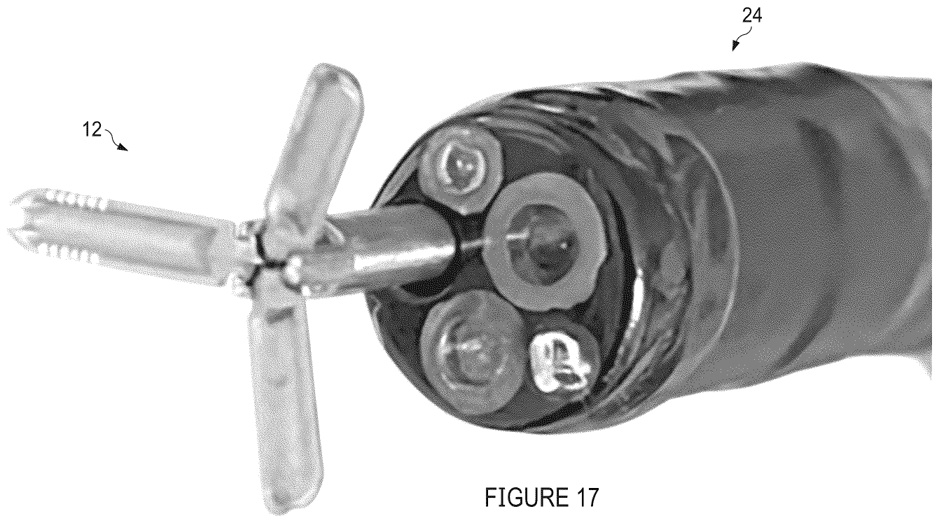

As illustrated with regard to FIG. 16 and FIG. 17, in exemplary embodiments, without limitation, the device 10 may be configured for use with an endoscope 24. For example, without limitation, the device 10, or at least certain components thereof (e.g., grasping portion 12 and at least part of the elongate structure 16) may be configured to fit through an internal passageway 70 (e.g., channel) of the endoscope 24, which may be a standard 2.8 mm through hole, by way of non-limiting example.

Alternatively, or additionally, one or more indicators 68, such as radiopaque markers or visible markings (e.g., contrasting colors, highlighted area, fluorescent material), may be provided at the grasping portion 12, such as but not limited to at a distal end of each of the jaws 38, such as to determine device 10 position and/or status during use, such as in combination with various imaging techniques. Such indicators 68 may be visible with imaging, such as from the endoscope 24, and/or otherwise provided, such as by separate imaging machines.

In exemplary embodiments, without limitation, an energy source 74 may be provided, such as at the handle 14 or separately therefrom. The energy source 74 may comprise, by way of non-limiting example, one or more batteries, external power supplies, radio frequency (RF) generators, combinations thereof, or the like. This energy may be transmitted, such as in a controlled fashion, to the grasping portion 12 using the mechanical linkage 56, particularly where the mechanical linkage 56 comprises one or more energy (e.g., thermal, RF) conductive materials, such as but not limited to, metal wire. This may allow the object being captured to be heated, by way of example. In the case of certain captured objects, this may allow for partial or complete object break down, cauterization, cutting, combinations thereof, or the like.

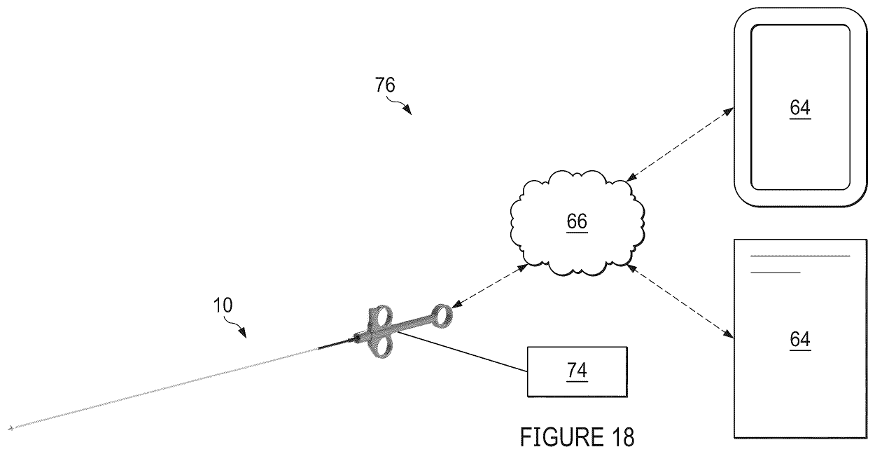

As illustrated with particular regard to FIG. 18, the device 10 may be part of a system 76. Some or all of the displays 60 may be remote from the device 10, and may be part of, or comprise, one or more remote electronic devices 64, such as but not limited to a smartphone, electronic display, tablet, computer, combinations thereof, or the like. Alternatively, or additionally, the data may be stored at one or more remote electronic devices 64, such as but not limited to database(s) and/or server(s) for later access. Connection to such devices 64 may be made by way of one or more networks 66 (e.g., internet, intranet, cellular network, local area network, near field connection, combinations thereof, or the like).

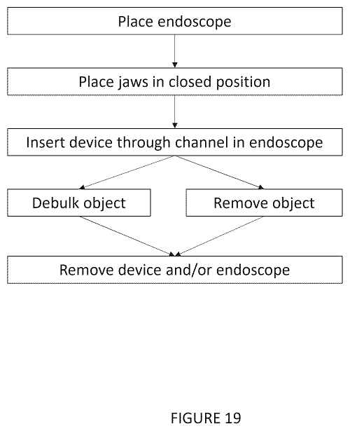

As illustrated with particular regard to FIG. 19, in exemplary embodiments, without limitation, the jaws 38 may be placed in the closed position and a distal end of the device, including the grasping portion 12 and at least part of the elongate structure 16, is inserted through the internal passageway 70 of the endoscope 24 adjacent to the food bolus or other structure of interest (“object”).

The endoscope 24 is preferably pre-placed, such as by way of a bite block in the patient's mouth, though such is not necessarily required. The endoscope 24 may be gently (e.g., under 10 lbs. of force) advanced beyond the epiglottis and into the hypopharynx and then beyond the upper esophageal sphincter, by way of example.

The endoscope 24 may be advanced into the esophagus until the object is visualized. The endoscope 24 may be advanced beyond the object, such as if what is visualized beyond it and then withdrawal the endoscope 24 proximal to the object, such as to try to advance it to the stomach with gentle pressure.

If advancing into the stomach is unavailable, fails, or otherwise undesirable, the device 10 may be utilized for manipulating the object, such as by debulking and/or removal. If the object is capable of being grasped sufficiently for removal, that may be preferred. If not, the object may be debulked and, in sections, advanced to the stomach (preferable), or removed.

In exemplary embodiments, without limitation, the jaws 38 may be opened, such as by manipulating the control element 58, and the object may be debulked.

Preferably, portion of the captured object within the jaws 38 is pushed into the stomach. Alternatively, or additionally, portions of the captured object within the jaws 38 is removed, such as through the internal passageway 70 and out of the patient's body, or with removal of the endoscope 24.

Further debulking and/or removal may be performed as needed, such as until the entire, or substantially all (e.g., at least 90%) of the object is removed or passed into the stomach by way of non-limiting example.

While food bolus removal is an exemplary application and use of the device 10, the device 10 may be used for other medical and/or non-medical procedures. For example, without limitation, while object removal (e.g., food bolus) is sometimes shown and/or discussed, the disclosed device 10, systems, and/or methods may be used in other applications, such as with other types and kinds of objects or tissues, endoscopic necrosectomy and debridement in pancreatic necrosis or walled-off pancreatic necrosis (WOPN), biopsies, polyplectomy, combinations thereof, or the like, such as in or by way of the esophagus, or other parts of the body, such as in a human or animal patient.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention.

Certain operations described herein may be performed by one or more electronic devices. Each electronic device may comprise one or more processors, electronic storage devices, executable software instructions, combinations thereof, and the like configured to perform the operations described herein. The electronic devices may be general purpose computers or specialized computing devices. The electronic devices may comprise personal computers, smartphones, tablets, databases, servers, or the like. The electronic connections and transmissions described herein may be accomplished by one or more wired or wireless connectively components (e.g., routers, modems, ethernet cables, fiber optic cable, telephone cables, signal repeaters, and the like) and/or networks (e.g., internets, intranets, cellular networks, the world wide web, local area networks, and the like). The computerized hardware, software, components, systems, steps, methods, and/or processes described herein may serve to improve the speed of the computerized hardware, software, systems, steps, methods, and/or processes described herein. The electronic devices, including but not necessarily limited to the electronic storage devices, databases, controllers, or the like, may comprise and/or be configured to hold, solely non-transitory signals.

### Statements

Statement 1. A multi-jaw grasper, said grasper comprising:
a handle comprising a control element;
an elongate structure having a proximal end portion extending from the handle;
a grasping portion located at a distal end portion of the elongate structure and comprising attachment segments and jaws, wherein each of said attachment segments is connected to a proximal end portion of a different set of two of the jaws, wherein the proximal end portion of each of said jaws contacts one another at a common location between the attachment segments, and wherein each of the jaws extends longitudinally outward from the common location; and
a linkage extending from the control element of the handle, within the elongate structure, and to the grasping portion where the linkage is connected to the proximal end portion of at least one of the jaws such that movement of the control element causes movement of the jaws between a closed position where jaws extend alongside one another and an opened position where a distal end of the jaws are splayed apart.

Statement 2. The grasper of statement 1, further comprising:
recesses located at each of the attachment segments; and
protrusions located at opposing sides of the proximal end portion of each of the jaws and mated with the recesses.

Statement 3. The grasper of any one of statements 1-2, wherein:
the protrusions of adjacent ones of the jaws contact one another.

Statement 4. The grasper of statement 3, wherein:
the recesses of a given one of the attachment segments are connected.

Statement 5. The grasper of any one of statements 1-4 wherein:
each of the jaws comprises a concave interior surface, and serrations on side edges thereof.

Statement 6. The grasper of any one of statements 1-5 wherein:
each of the jaws comprise spaced apart tips at the distal end thereof forming a channel therebetween.

Statement 7. The grasper of statement 6 wherein:
the jaws form an enclosed capsule when in the closed position, aside from the channels.

Statement 8. The grasper of any one of statements 1-5 wherein:
each of the jaws comprise a curved tip at the distal end thereof forming openings on either side of the tip.

Statement 9. The grasper of statement 8 wherein:
the side edges of the jaws come together when in the closed position, such as to form an enclosed capsule aside from the openings.

Statement 10. The grasper of any one of statements 1-9 wherein:
the proximal end portion of each of the jaws comprise a rounded surface, where the rounded surface of each of the jaws contacts one another at the common location.

Statement 11. The grasper of statement 10 wherein:
the proximal end portion of each of the jaws comprises opposing recessed areas defining, at least in part, a ridge forming at least part of, or extending from, the rounded surface.

Statement 12. The grasper of statement 11 further comprising:
a through hole extending through the ridge of a respective one of the jaws to connect the recessed areas, wherein the linkage comprise a wire extended through the through hole.

Statement 13. The grasper of statement 12 wherein:
each of the jaws comprises one of the through holes;
the linkage comprise multiple wires, each extended through one of the through holes of a respective one of the jaws.

Statement 14. The grasper of any one of statements 12-13 further comprising:
a ball-shaped tip provided at a distal end of the wire and sized to fictionally engage the through hole upon retraction of the wire.

Statement 15. The grasper of any one of statements 13-14 wherein:
each of the wires, after extending through the through hole of the respective one of the jaws, loops back and is secured to itself.

Statement 16. The grasper of any one of statements 1-15 wherein:
the linkage comprises a first portion with multiple wires extending alongside one another and/or connected to one another and a second portion whereby the multiple wires are spaced apart, at least along a length thereof, to each connect to a respective one of the jaws.

Statement 17. The grasper of any one of statements 1-16 wherein:
the attachment segments are defined, at least in part, by openings separating the attachment segments; and
the attachment portion includes a common portion interconnecting the attachment segments.

Statement 18. The grasper of statement 17 further comprising:
a sleeve extending over at least a portion of the openings.

Statement 19. The grasper of statement 18 wherein:
the common portion comprises a base portion which is recessed relative to the attachment segments to receive the sleeve.

Statement 20. The grasper of statement 18 wherein:
the sleeve is adhered to the attachment portion.

Statement 21. The grasper of any one of statements 17-20 wherein:
the attachment portion comprises a base portion secured to, and at least partially within, the distal end of the elongate structure and comprising one or more channels extending therethrough; and
the linkage extends through the one or more channels.

Statement 22. The grasper of statement 21 wherein:
the one or more channels comprise three channels, spaced apart from one another and located adjacent to an outer edge of the base portion;
the linkage comprise three distinct distal ends, each extending through one of the three channels;
the jaws include three jaws; and
each of the three distinct distal ends of the linkage is connected to a different one of the three jaws.

Statement 23. The grasper of statement 22 wherein:
the linkage comprises a braided wire having the three distinct distal ends, each comprising one of the wires in the braided wire.

Statement 24. The grasper of statement 22 wherein:
the linkage comprises three distinct, unbraided wires.

Statement 25. The grasper of statement 22 wherein:
each of the three distinct ends of the linkage extend through a respective one of the three channels opposing the respective one of the jaws to which the respective distinct end of the linkage is attached.

Statement 26. The grasper of statement 25 wherein:
each of the three distinct ends of the linkage is attached to a distal end portion of the respective one of the jaws.

Statement 27. The grasper of statement 26 wherein:
a proximal end of each of the jaws is curved and comprises a groove along an exterior surface thereof to accommodate one of the three distinct ends of the linkage and a hole extending to an interior of the jaw.

Statement 28. The grasper of statement 27 wherein:
the distal end portion of each of the jaws includes a recess which receives a respective one of the three distinct ends of the linkage.

Statement 29. The grasper of statement 28 wherein:
the recesses extend to an exterior of the jaws.

Statement 30. The grasper of statement 29 wherein:
the recesses are shaped to extend towards the proximal ends of the jaws.

Statement 31. The grasper of any one of statements 1-30 wherein:
the grasping portion comprises three of the jaws and three of the attachment segments.

Statement 32. The grasper of any one of statements 1-31 wherein:
the linkage comprises a wire attached to a single one of the jaws.

Statement 33. The grasper of any one of statements 1-32 wherein:
the linkage is connected to each of the jaws.

Statement 34. The grasper of any one of statements 1-33 further comprising:
an energy source located at the handle and in electric connection with the linkage, wherein the linkage comprises an energy conductive material.

Statement 35. The grasper of any one of statements 1-34 wherein:
the handle comprises loops for grasping, at least one of which is moveable and provides the control element.

Statement 36. The grasper of any one of statements 1-35 wherein:
at least the elongate structure and the grasping portion, when in the closed position, are configured to fit within, and move relative to, an internal passageway of an endoscope.

Statement 37. The grasper of any one of statements 1-36 wherein:
one or more visible indicators provided at one or more of the jaws.

Statement 38. The grasper of any one of statements 1-37 wherein:
one or more sensors provided at one or more of the jaws.

Statement 39. A method of using the grasper of any one of statements 1-38, said method comprising:
placing the jaws in the closed position;
advancing the grasping portion through an internal passageway of an endoscope positioned within an esophagus of a patient in proximity to an object for removal such that at least the jaws of the grasper are exposed from a distal end of the endoscope and at least the handle is exposed from a proximal end of the endoscope; and
debulking and/or removing the object using the jaws.

Statement 40. A system comprising:
the grasper of any one of statements 1-39; and
an endoscope comprising an internal passageway sized to accommodate the grasping portion when the jaws and in the closed position, and the elongate structure of the grasper such that the grasping portion, with the jaws in the closed position, is insertable through a proximal end of the endoscope and at least the jaws of the grasping portion are extendable beyond a distal end of the endoscope for movement into the opened position while at least the handle remains exposed from a proximal end of the endoscope.

What is claimed is:

1. A multi-jaw grasper, said grasper comprising:
a handle comprising a control element;
an elongate structure having a proximal end portion extending from the handle;
a grasping portion located at a distal end portion of the elongate structure and comprising attachment segments and jaws, wherein each of said attachment segments is connected to a proximal end portion of a different set of two of the jaws, wherein the proximal end portion of each of said jaws contacts one another at a common location between the attachment segments, and wherein each of the jaws extends longitudinally outward from the common location; and
a linkage extending from the control element of the handle, within the elongate structure, and to the grasping portion where the linkage is connected to the proximal end portion of at least one of the jaws such that movement of the control element causes movement of the jaws between a closed position where jaws extend alongside one another and an opened position where a distal end of the jaws are splayed apart.

2. The grasper of claim 1, further comprising:
recesses located at each of the attachment segments, wherein the recesses of a given one of the attachment segments are connected; and
protrusions located at opposing sides of the proximal end portion of each of the jaws and mated with the recesses, wherein the protrusions of adjacent one of the jaws contact one another.

3. The grasper of claim 1 wherein:
each of the jaws comprises a concave interior surface, and serrations on side edges thereof.

4. The grasper of claim 1 wherein:
each of the jaws comprise spaced apart tips at the distal end thereof forming a channel therebetween; and
the jaws form an enclosed capsule when in the closed position, aside from the channels.

5. The grasper of claim 1 wherein:
each of the jaws comprise a curved tip at the distal end thereof forming openings on either side thereof; and
side edges of the jaws come together when in the closed position.

6. The grasper of claim 1 wherein:
the proximal end portion of each of the jaws comprise a rounded surface, where the rounded surface of each of the jaws contacts one another at the common location.

7. The grasper of claim 6:
wherein the proximal end portion of each of the jaws comprises opposing recessed areas defining, at least in part, a ridge forming at least part of, or extending from, the rounded surface;
further comprising a through hole extending through the ridge of a respective one of the jaws to connect the recessed areas; and
wherein the linkage comprise a wire extended through the through hole.

8. The grasper of claim 7 wherein:
the attachment portion comprises a base portion secured at least in part within the distal end of the elongate structure and comprising three channels extending therethrough;
the channels are spaced apart from one another and are located adjacent to a outer edge thereof;
the linkage includes three distinct distal ends, each extending through one of the three channels;
the jaws include three jaws; and
each of the three distinct distal ends of the linkage is connected to a different one of the three jaws.

9. The grasper of claim 8 further comprising:
a ball-shaped tip provided at each of the three distinct ends of the wire and sized to fictionally engage the through hole upon retraction of the wire.

10. The grasper of claim 1 wherein:
the proximal end portion of each of the jaws comprise a rounded surface having a hole extending from the rounded surface to an interior of the jaw, where the rounded surface of each of the jaws contacts one another at the common location.

11. The grasper of claim 10 wherein:
the attachment portion comprises a base portion secured at least in part within the distal end of the elongate structure and comprising three channels extending therethrough which are spaced apart from one another and are located adjacent to a outer edge thereof; and
the linkage includes three distinct distal ends, each extending through one of the three channels to a different one of the jaws positioned distal from the respective one of the channels and is attached to a distal end portion of the respective one of the jaws.

12. The grasper of claim 11 wherein:
each of the jaws includes a groove at the rounded surface to accommodate one of the three distinct distal ends of the linkage; and
each of the jaws includes a recess for the respective one of the three distinct distal ends at the interior of the respective jaw which extends to an outer surface of the respective jaw.

13. The grasper of claim 1 wherein:
the grasping portion comprises an attachment portion comprising:
attachment segments defined, at least in part, by openings separating the attachment segments; and
a common portion interconnecting the attachment segments.

14. The grasper of claim 13 wherein:
the grasping portion comprises three of the jaws and three of the attachment segments.

15. The grasper of claim 14 further comprising:
a sleeve extending over at least a portion of each of the openings.

16. The grasper of claim 1 wherein:
the linkage comprises a wire attached to a single one of the jaws.

17. The grasper of claim 1 further comprising:
an energy source located at the handle and in electric connection with the linkage, wherein the linkage comprises an energy conductive material.

18. The grasper of claim 1 wherein:
at least the elongate structure and the grasping portion, when in the closed position, are configured to fit within, and move relative to, an internal passageway of an endoscope.

19. A method of using the grasper of claim 18, said method comprising:
placing the jaws in the closed position;
advancing the grasping portion through the internal passageway of the endoscope positioned within an esophagus of a patient in proximity to an object for removal such that at least the jaws of the grasper are exposed from a distal end of the endoscope and at least the handle is exposed from a proximal end of the endoscope; and
debulking and/or removing the object using the jaws.

20. A system comprising:
the grasper of claim 1; and
an endoscope comprising an internal passageway sized to accommodate the grasping portion when the jaws and in the closed position, and the elongate structure of the grasper such that the grasping portion, with the jaws in the closed position, is insertable through a proximal end of the endoscope and at least the jaws of the grasping portion are extendable beyond a distal end of the endoscope for movement into the opened position while at least the handle remains exposed from a proximal end of the endoscope.
